(19) United States
(12) Patent Application Publication
(10) Pub. No.: US 2023/0302263 A1
(43) Pub. Date: Sep. 28, 2023

(54) REMOVABLE DUAL FUNCTION LOADING TOOL & PROTECTOR FOR DRUG COATED MEDICAL DEVICES

(71) Applicant: Cook Medical Technologies LLC, Bloomington, IN (US)

(72) Inventors: John Savage, Dublin (IE); Thomas Mindel, Bloomington, IN (US); Michael Sayers, Co. Tipperary (IE); Joe Dervan, Co. Galway (IE); James Butler, Co. Tipperary (IE); Eugene Folan, Co. Clare (IE); Alec Cerchiari, Bloomington, IN (US); Aaron Reid, Co. Tipperary (IE)

(73) Assignee: Cook Medical Technologies LLC, Bloomington, IN (US)

(21) Appl. No.: 18/159,970

(22) Filed: Jan. 26, 2023

Related U.S. Application Data
(60) Provisional application No. 63/359,028, filed on Jul. 7, 2022.
(60) Provisional application No. 63/303,736, filed on Jan. 27, 2022.

(51) Int. Cl. A61M 25/10

(52) U.S. Cl. CPC A61M 25/1029; A61M 2025/1081

(57) ABSTRACT

A medical device assembly including a catheter having a proximal end, a distal end, and a lumen therethrough. A medical device is disposed about a distal portion of the catheter. A removable loading tool and protector is disposed about the catheter and the medical device, and includes a body having a generally tubular configuration and having a proximal end, a distal end, an intermediate region disposed between the proximal end and the distal end, and a lumen disposed therethrough; and a proximal region having a length and extending from the proximal end of the body, the proximal region having at least one slit positioned adjacent to the proximal end of the body. The proximal region has a first inner diameter and the intermediate region has a second inner diameter, the first inner diameter of the proximal region being greater than the second inner diameter of the intermediate region.

### RELATED APPLICATIONS

This application claims the benefit of U.S. Provisional Application No. 63/359,028, filed Jul. 7, 2022, and U.S. Provisional Application No. 63/303,736, filed Jan. 27, 2022, each of which are hereby incorporated herein by reference in their entirety.

### TECHNICAL FIELD

The disclosure relates generally to a medical device delivery system. More specifically, this disclosure relates to a removable dual function loading tool and balloon protector for drug coated medical devices and uses thereof.

### BACKGROUND

Local delivery of a therapeutic agent can be useful in the treatment of many medical conditions. Illustratively, local delivery of a therapeutic agent within a body vessel or to a selected portion of internal body tissue can eliminate or reduce the need for systemic delivery of the therapeutic agent thus minimizing any potential adverse effect of the therapeutic agent on areas of the body not needing treatment.

Minimally invasive implantable medical devices, such as balloons, catheters and stents, can provide a platform for delivering therapeutic agents to internal body tissue. For example, balloon catheters or stents may be used to deliver a therapeutic agent directly to the target site within a body vessel such as an artery or vein.

One example of a condition that can be beneficially treated by local administration of a therapeutic agent with a balloon catheter is the delivery of a therapeutic agent in combination with percutaneous transluminal coronary angioplasty (PTCA), a technique used to dilate stenotic portions of blood vessels. Although PTCA and related procedures aid in alleviating intraluminal constrictions, such constrictions or blockages may reoccur in many cases. The cause of these recurring obstructions, termed restenosis, may be due to the body responding to the surgical procedure. Restenosis of the vessel may develop over several months after the procedure, and may require another angioplasty procedure or a surgical bypass operation to correct.

Proliferation and migration of smooth muscle cells (SMC) from the media layer of the lumen to the intimal layer cause an excessive production of extracellular matrices (ECM), which is believed to be one of the leading contributors to the development of restenosis. The extensive thickening of tissues narrows the lumen of the blood vessel, constricting or blocking the blood flow through the vessel.

Drugs that inhibit restenosis may be locally delivered during PTCA from a balloon catheter or by placement of a stent configured to continue to release the drug after the PTCA procedure. The delivery of the drug from coatings in these and other minimally invasive procedures can be complicated by the need to have a coating that is durable during delivery, but which effectively delivers the drug when implanted in the region where local treatment is desired.

Delivery of a therapeutic agent through the use of a balloon catheter may be delivered into a patient through the use of an access sheath or guide sheath in combination with a hemostatic valve. Thus, there is a desire to minimize the amount of damage that may occur to the balloon or to the coating of therapeutic agent, in order to maintain the therapeutic usability of the device. Furthermore, there is a desire to improve the working length of the balloon catheter.

### SUMMARY OF THE INVENTION

The present disclosure refers generally to a medical device delivery system. More specifically, this disclosure relates to a removable dual function loading tool and balloon protector for drug coated medical devices and uses thereof.

In one aspect, a removable loading tool and protector for a medical device includes a body having a generally tubular configuration and having a proximal end, a distal end, an intermediate region disposed between the proximal end and the distal end and a lumen disposed therethrough. The removable loading tool and protector for a medical device includes a proximal region having a length and extending from the proximal end of the body, the proximal region comprising at least one slit positioned adjacent to the proximal end of the body. The proximal region has a first inner diameter and the intermediate region has a second inner diameter, the first inner diameter of the proximal region being greater than the second inner diameter of the intermediate region. In some embodiments, the first inner diameter of the proximal region is uniform throughout its length. In alternative embodiments, the proximal region comprises a first section and a tapered second section, the tapered second section positioned distal to the uniform first section.

In another aspect, a medical device assembly includes a catheter having a proximal end, a distal end, and a lumen therethrough. A drug coated expandable medical device disposed about a distal portion of the catheter, the expandable medical device having a distal end and a proximal end. A loading tool and balloon protector is disposed about the catheter and the expandable medical device, the loading tool and protector includes a body having a generally tubular configuration, and having a proximal end, a distal end, and an intermediate region disposed between the proximal end and the distal end and having a a lumen disposed therethrough. The loading tool and balloon protector includes a proximal region having a length and extending from the proximal end of the body, the proximal region comprising a slit positioned adjacent to the proximal end of the body. The proximal region has a first diameter and the intermediate region has a second diameter, the first diameter of the proximal region being greater than the second diameter of the intermediate region. In some embodiments, the distal end has a beveled configuration having an angle. In other embodiments, a rib is disposed about an outer surface of the body proximate to the distal end.

In yet another aspect, a medical device assembly includes a catheter having a proximal end, a distal end, and a lumen therethrough. A drug coated expandable balloon is disposed about a distal portion of the catheter, the expandable balloon having a distal end and a proximal end. A loading tool and protector is removably disposed about the catheter and the expandable balloon, the loading tool and protector includes a body having a generally tubular configuration and having a proximal end, a beveled distal end, an intermediate region disposed between the proximal end and the distal end and a lumen disposed therethrough; and a proximal region having a length and extending from the proximal end of the body, the proximal region comprising a first section, a tapered second section, and at least one slit positioned adjacent to the proximal end of the body and within the first section, the slit having a length. The first section of the proximal region has a first diameter and the intermediate region has a second diameter, the first diameter of the first section of the proximal region being greater than the second diameter of the intermediate region. The length of the slit is less than the length of the proximal region.

Further areas of applicability will become apparent from the description provided herein. It should be understood that the description and specific examples are intended for purposes of illustration only and are not intended to limit the scope of the present disclosure.

### BRIEF DESCRIPTION OF DRAWINGS

In order that the disclosure may be well understood, there will now be described various forms thereof, given by way of example, reference being made to the accompanying drawings. The components in each of the drawings may not necessarily be drawn to scale, rather emphasis is placed upon illustrating the principles of the invention. Moreover, like referenced numerals in different drawings designate corresponding or similar components or elements.

FIG. 1 is an illustration of an embodiment of a drug delivering balloon catheter having an embodiment of a removable dual function loading tool and medical device protector according to the teachings of the present disclosure.

FIG. 2 is an illustration of a cross sectional view of an embodiment of a balloon mounted region of the balloon catheter according to the teachings of the present disclosure.

FIG. 3 illustrates an embodiment of a removable dual function loading tool and medical device protector according to the teachings of the present disclosure.

FIG. 4 illustrates a perspective view of an embodiment of a removable dual function loading tool and medical device protector according to the teachings of the present disclosure.

FIG. 5 illustrates an embodiment of a distal region of the drug delivering balloon catheter and the removable dual function loading tool and medical device protector according to the teachings of the present disclosure.

FIG. 6 illustrates an embodiment of the distal region of the drug delivering balloon catheter and the removable dual function loading tool being positioned within an embodiment of an access sheath or guiding sheath having a hemostatic valve according to the teachings of the present disclosure.

FIGS. 7A and 7B illustrate a proximal end of the removable dual function loading tool and medical device protector according to the teachings of the present disclosure.

FIGS. 8A and 8B illustrate positioning of a distal tip of a drug delivering balloon catheter within an embodiment of a removable dual function loading tool and medical device protector according to the teachings of the present disclosure.

The drawings described herein are for illustration purposes only and are not intended to limit the scope of the present disclosure in any way.

### DETAILED DESCRIPTION

Example embodiments are disclosed herein. It is understood, however, that the disclosed embodiments are merely exemplary and may be embodied in various and alternative forms. The figures are not necessarily to scale; some figures may be configured to show the details of a particular component. Therefore, specific structural and functional details disclosed herein are not to be interpreted as limiting but merely as a representative basis for the claims and/or teaching one skilled in the art to practice the embodiments.

Unless defined otherwise, all technical and scientific terms used herein have the same meaning as commonly understood by one of ordinary skill in the art to which this invention pertains.

The terms “patient,” “subject,” and “recipient” as used in this application may refer to any animal, particularly humans.

The term “proximal” is used to refer to the end of the medical device (or component thereof) that is closest to the operator during use of the system. The term “distal” is used to refer to the end of the medical device (or component thereof) that is initially inserted into the patient, or that is closest to the patient during use.

The term “biocompatible” refers to a material that is substantially non-toxic in the in vivo environment of its intended use, and that is not substantially rejected by the patient’s physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part 1: Evaluation and Testing.” Typically, these tests measure a material’s toxicity, infectivity, pyrogenicity, irritation potential, reactivity, hemolytic activity, carcinogenicity and/or immunogenicity. A biocompatible structure or material, when introduced into a majority of patients, will not cause a significantly adverse, long-lived or escalating biological reaction or response, and is distinguished from a mild, transient inflammation which typically accompanies surgery or implantation of foreign objects into a living organism.

The term “medical device” means any object that is itself or that includes a component that is intentionally inserted into the body of a patient as part of a medical treatment, and that comprises a structure adapted for introduction into a patient. The medical device can be a tool, such as, without limitation, a catheter, a wire guide, a forceps, or a scissors used to affect a surgical procedure at and/or deliver a second medical device to a treatment site in a patient.

The terms “about” and “substantially” are used herein with respect to measurable values and ranges due to expected variations known to those skilled in the art (e.g., limitations and variability in measurements).

The terms “at least one” and “one or more of” an element are used interchangeably and may have the same meaning. These terms, which refer to the inclusion of a single element or a plurality of the elements, may also be represented by the suffix “(s)” at the end of the element. For example, “at least one metal”, “one or more metals”, and “metal(s)” may be used interchangeably and are intended to have the same meaning.

The term “therapeutic effect” as used herein means an effect which induces, ameliorates or otherwise causes an improvement in the pathological symptoms, disease progression or physiological conditions associated with or resistance to succumbing to a disorder, for example restenosis, of a human or veterinary patient. The term “therapeutically effective amount” as used with respect to a drug means an amount of the drug which imparts a therapeutic effect to the human or veterinary patient.

The term “excipient” as used herein refers to a compound that speeds the release of the drug from the coated device and/or increases the amount of the drug released from the device when the device is implanted in the body of a patient.

The present disclosure refers generally to a medical device delivery system. More specifically, this disclosure relates to a removable dual function loading tool and medical device protector for drug coated medical devices and uses thereof. Further, the removable dual function loading tool and medical device protector may be used in conjunction with, and packaged with, drug delivering medical devices, such as a balloon and a balloon catheter. The removable dual function loading tool and medical device protector may be used to introduce the drug delivering medical device into an access or guiding sheath, particularly one that may include a hemostatic valve. A hemostatic valve may cause a shearing effect to the coating or the medical device itself, which may damage coating uniformity and may cause unforeseen implications from a therapeutic standpoint. Accordingly, the removable dual function loading tool and medical device protector allows for passage of the drug coated medical device through the hemostatic valve while minimizing or eliminating damage to the balloon or the drug coating. Additionally, there may be an improvement in the quantity of drug coated material that is delivered to the target site within the patient. Further, as the dual function loading tool and balloon protector is removable, the working length available of the balloon catheter may be maximized.

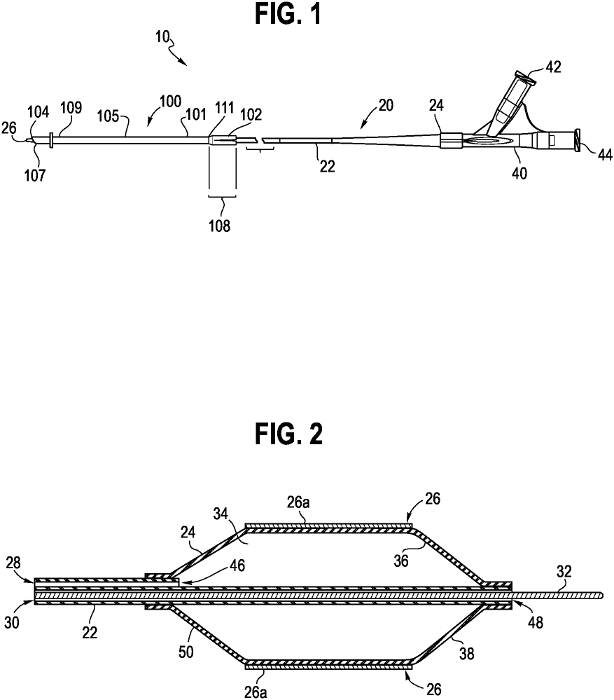

Referring to the figures, an embodiment of a drug delivering medical device assembly 10 and an embodiment of a dual function loading tool and removable medical device protector 100 useful in performing minimally invasive surgery in the treatment of a medical condition is provided. As shown in FIG. 1, the medical device assembly 10 includes a balloon catheter 20. The balloon catheter includes a catheter shaft 22 having a proximal end 24 and a distal end 26 and a balloon (not shown) positioned on a balloon receiving region of the shaft 22. The balloon catheter 20 may also include a catheter hub 40 mounted to shaft 22. The catheter hub 40 defines a first opening 42 and a second opening 44. While the present embodiment is directed to a balloon catheter 20, other medical devices, such as self-expanding or balloon expandable stents and delivery systems for implanting valves and catheters, may also be utilized in view with the teachings of the present disclosure.

To facilitate introduction of the balloon catheter 20 through an access or guiding sheath having a valve, such as a hemostatic valve, on its proximal end and into the body of a patient, a removable dual function loading tool and medical device protector 100 is included. The removable dual function loading tool and medical device protector 100 has a body 101 having a proximal region 108 including a proximal end 102 and a distal region 109 including a distal end 104 and an intermediate region 105 disposed between the proximal region 108 and the distal region 109. As shown, a boundary 111 is present to delineate between the proximal region 108 and the intermediate region 105 of the body 101 of the removable dual function loading tool and medical device protector. The proximal region 108 includes a slit 116 having a length that extends from the proximal end 102 of the body 101 of the removable dual function loading tool and medical device protector 100. As shown, the slit 116 terminates prior to the boundary 111 and the intermediate region 105.

The body 101 may have a generally tubular configuration with a lumen 107 disposed therethrough. The lumen 107 may be configured to receive a medical device, such as a balloon. In some embodiments, a mandrel (not shown) may be positioned within the lumen 107 of the removable dual function loading tool and medical device protector 100 in order to support the device when it is packaged. The mandrel may be manufactured from suitable material, including, but not limited to, stainless steel. The mandrel is removed prior to use of the balloon catheter 20 and the removable dual function loading tool and medical device protector 100.

As shown, the removable dual function loading tool and medical device protector 100 is disposed about over the balloon (not shown) on a distal region of the catheter shaft 22. The removable dual function loading tool and balloon protector 100 may be independently moveable with respect to the catheter shaft 22 of the balloon catheter 20, which may allow the distal end 26 of the balloon catheter 20 to remain flush with the distal end 104 of the removable dual function loading tool and balloon protector 100 or to extend distal of the distal end 104 of the removable dual function loading tool and balloon protector 100, as shown in FIGS. 1, 8A, and 8B. This freedom of movement may also provide a visual cue to the user of the medical device assembly 10 when it is being positioned for use. In some embodiments, for example, as shown in FIG. 1, a depth controlling rib 106 is positioned proximate to the distal end 104 of the removable dual function loading tool and medical device protector 100.

The proximal region 108 extends from the proximal end 102 of the body 101 removable dual function loading tool and medical device protector 100. As will be discussed further below, the proximal region 108 allows for proximal to distal loading of the balloon within the removable dual function loading tool and medical device protector 100. This feature also allows for the removable dual function loading tool and medical device protector 100 to be removably packaged with the balloon catheter 20 and positioned over the balloon during manufacturing to protect the balloon from damage, which advantageously increases usability and helps meets the requirements of packaging. This feature eliminates the necessity of having a loading tool for use with a pre-packaged balloon catheter and a separate balloon protector, thus reducing the possibility of waste.

FIG. 2 shows an embodiment of a balloon 24 for use with the balloon catheter 20. A material coat 26 including a layer containing a drug and excipient 26a as described herein is carried by balloon 24. The catheter shaft 22 may include a first lumen 28 and a second lumen 30. Lumen 28 is configured for inflation of the balloon 24 and is in fluid communication with the opening 42 of catheter hub 40. Lumen 30 is configured to receive a guide wire 32 or other guide member to be used in conjunction with the balloon catheter 20 and is in communication with the opening 44 of the catheter hub 40. The balloon 24 includes an interior region configured to receive a liquid or other fluid for the inflation of balloon 24. Balloon 24 has an inner wall 36 bounding balloon interior 34, and an outer wall surface 38. Layer 26 is adhered to outer wall surface 38 of balloon 24. Opening 42 of the catheter hub 40 and lumen 28 communicate with an opening 46 into the interior 34 of balloon 24, for passage of the inflation fluid for the balloon 24. Opening 44 of the catheter hub 40 and lumen 30 defined by the catheter shaft 22 extend to a distal opening 48 of lumen 30, with distal opening positioned distally of balloon 24. Although FIG. 2 depicts an example balloon structure, it may have any of the features known for such balloons, including a different shape, surface roughening, texturing and so on.

The drug will typically be incorporated in the balloon 24 in a therapeutically effective amount. In this regard, it will be understood that where the drug is a restenosis-inhibiting agent, the restenosis-inhibiting agent will be incorporated in the coating in an amount that is effective to inhibit restenosis when the implantable medical device (e.g. a balloon or stent) is deployed so as to deliver the drug from the implantable medical device to a wall of the artery, vein or other vessel or passage that is being treated by the device. As will be recognized, the level of a drug that will be therapeutically effective will vary in accordance with the particular drug in use, the implantable medical device in use, the implant site, the condition to be treated, the composition of the coating including the drug, and other potential factors. Through routine experimentation, the achievement of a therapeutically effective amount of drug will be within the purview of those of ordinary skilled in the field.

The excipient can be included in the device in an amount effective to increase the rate of release of the drug from the device at a site of implant of the implantable medical device structure (as compared to the rate of release from an otherwise identical device not including the excipient). In other embodiments, the excipient is present in an amount that increases the amount of drug released from the device as compared to an otherwise identical device not including the excipient.

The balloon 24 of the balloon catheter 20 may have a balloon wall made of any suitable balloon wall material, typically a polymeric balloon wall material. The polymeric or other balloon wall material can be elastomeric, as in the case of an illustrative silicone elastomer, latex rubber elastomer, nylon elastomer, or polyurethane elastomer balloon film, where the balloon can expand upon inflation due to the expansion and thinning of the balloon wall material. The compliance of the balloon wall material in such elastomeric balloon applications is typically greater than 20% and more typically greater than 50%, and/or the burst pressure of such elastomeric balloons will typically be in the range of about 1.1 to about 2 atmospheres. In other embodiments, the polymeric or other balloon wall material can be inelastic, as in the case of a non-compliant or semi-compliant balloon (e.g. as commonly used in angioplasty and/or stent delivery balloons), where the balloon can expand upon inflation due to the unfolding of the balloon wall material from an initial folded configuration. Preferred balloon wall materials for non-compliant or semi-compliant balloons include polyamide (e.g. as in Nylon balloons), polyethylene terephthalate (PET), or polyurethane polymers. At least a portion of and potentially the entirety of such an elongate, generally cylindrical outer surface can carry the drug and excipient as discussed herein, either as the sole coating carried by the generally cylindrical outer surface or in combination with one or more additional coatings carried by the generally cylindrical outer surface.

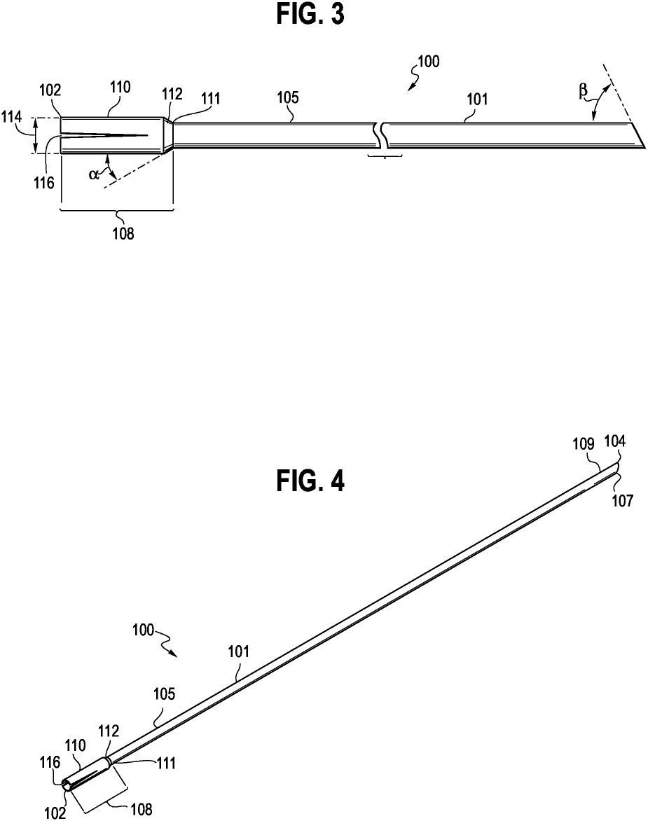

FIGS. 3 and 4 illustrate embodiments of a removable dual function loading tool and medical device protector 100 according to the teachings of the present disclosure. The removable dual function loading tool and medical device protector 100 includes a body 101 having a proximal region 108 including a proximal end 102, a distal region 109 including a distal end 104, an intermediate region 105 disposed between the proximal region 108 and the distal region 109, and a lumen 107 disposed therein. A boundary 111 may be present to delineate between the proximal region 108 and the intermediate region of the body 101 of the removable dual function loading tool and medical device protector 100. The intermediate region 105 includes an outer diameter and an inner diameter and may range from 4 Fr to 7 Fr. A proximal region 108 extends from the proximal end 102 of the body 101 of the removable dual function loading tool and medical device protector 100. The proximal region 108 includes a first section 110 and a tapered second section 112. As shown, the first section 110 has a first inner diameter 114. The first inner diameter 114 of the first section 110 is configured to be greater than an inner diameter of the intermediate region 105. This difference in the inner diameter of the first section 110 and the inner diameter of the intermediate region 105 allows for easier loading of the balloon into the removable dual function loading tool and medical device protector 100 prior to packaging. The greater inner diameter of the first section 110 minimizes the possibility of any unnecessary shear to the balloon during loading. In addition, the first section 110 of the proximal region 108 provides a gripping area for the user when using the device.

The tapered second section 112 forms an angle α with respect to a longitudinal axis of the body 101 of the dual function loading tool and medical device protector 100. The angle α of the tapered second section 112 may be 45 degrees or less with respect to a longitudinal axis of the body 101. In a particular embodiment, the angle α of the tapered second section 112 may be 30 degrees with respect to a longitudinal axis of the body 101. As shown, the tapered second section 112 connects the proximal region 108 to the intermediate region 105. In addition, the inner diameter of the tapered second section 112 ranges from the inner diameter of the first section 110 to the inner diameter of intermediate region 105.

The proximal region 108 also includes at least one slit 116 having a length that extends from the proximal end 102 of the removable dual function loading tool and medical device protector 100. As shown, the slit 116 terminates proximal to the boundary 111 and beginning of the tapered second section 112 of the proximal region 108. The slit 116 allows for the removable dual function loading tool and balloon protector to be removed during use of the removable dual function loading tool and medical device protector 100. In some embodiments, the removable dual function loading tool and balloon protector 100 may include a slit 116 disposed on opposite sides of the proximal region 108.

During the loading of the balloon catheter 20 within the proximal region 108 having a slit 116, the proximal region 108 may collapse inward, which may cause misalignment of the two halves of the proximal region 108, reduction in the radial strength of the proximal region 108, and/or deformation in the configuration of the proximal region 108. When the balloon is a drug coated balloon, those structural disruptions in the proximal region 108 can cause the coated drug to be removed from the balloon surface during loading of the balloon. Thus, in some embodiments, during the loading of the balloon catheter 20 into the removable dual function loading tool and medical device protector 100, a device, such as a proximal region protector, may be used to maintain the radial strength and the configuration of the proximal region 108, for example, by maintaining the alignment of the first and second segments 120, 122 of the proximal section 108. Maintaining the radial strength and the configuration of the proximal region 108 during loading of the balloon catheter 20 reduces the risk of drug loss from the balloon surface due to misalignment and ensures that the drug coating on the balloon surface remains therapeutically effective for treating the patient. Once loaded, the proximal region protector may be removed and the medical device assembly 10 comprising the balloon catheter 20 disposed within the removable dual function loading tool and medical device protector 100 may be packaged for use.

The distal end 104 of the removable dual function loading tool and medical device protector 100 may be a beveled configuration, as shown in FIGS. 3 and 4. The beveled configuration provides sufficient strength to the distal end of the removable dual function loading tool and medical device protector 100 and also reduces the amount of force necessary to push the distal end 104 through a hemostatic valve of an access sheath. The angle β of the distal end 104 of the removable dual function loading tool and medical device protector 100 may range from 10 to 90 degrees. More particularly, the angle β of the distal end 104 of the removable dual function loading tool and medical device protector 100 may range from 30 and 45 degrees.

The removable dual function loading tool and medical device protector 100 may be manufactured from a polymeric material. The material of construction for the implantable medical device structure can be biodegradable or non-biodegradable. Nonbiodegradable polymers that can be used include, for example, cellulose acetate, cellulose nitrate, silicone, polyethylene terephthalate, polyurethane, polyamide, polyester (e.g. Nylon), polyorthoester, polyanhydride, polyether sulfone, polycarbonate, polypropylene, high molecular weight polyethylene, high density polyethylene, and polytetrafluoroethylene, or mixtures of these. Biodegradable polymers that can be used include, for example, polylactic acid (PLA), polyglycolic acid (PGA), poly(lactic-co-glycolic acid) (PLGA), polyanhydride, polycaprolactone, polyhydroxybutyrate valerate, or mixtures of these. In a particular example, the removable dual function loading tool and medical device protector 100 comprises polytetrafluoroethylene. In an alternative example, the removable dual function loading tool and medical device protector 100 comprises high density polyethylene. The removable dual function loading tool and medical device protector 100 may be configured to be peelable along its length.

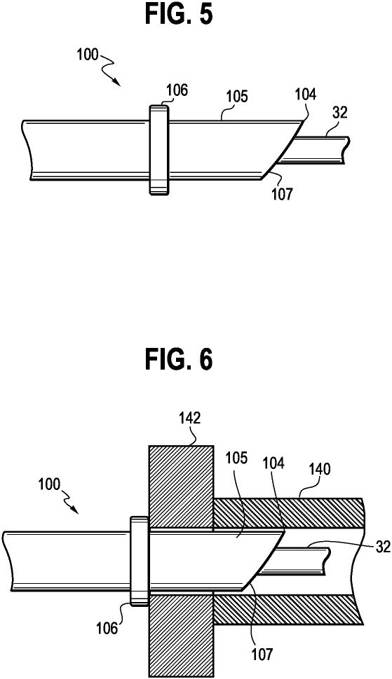

FIG. 5 illustrates an embodiment of a distal region of the removable dual function loading tool and removable balloon 100. As shown, the distal end 104 of the removable dual function loading tool and medical device protector 100 has a beveled configuration. Extending beyond the distal end 104 of the removable dual function loading tool and medical device protector 100 is a guide wire 32. In some embodiments, a depth controlling rib 106 may be provided proximate to the distal end 104 of the removable dual function loading tool and medical device protector 100. The depth controlling rib 106 allows the user to control the depth the removable dual function loading tool and medical device protector 100 as it is pushed through a hemostatic valve of an access sheath. The depth controlling rib 106 also acts as a restraint to prevent the removable dual function loading tool and medical device protector 100 from being pushed fully into an access sheath.

FIG. 6 illustrates an embodiment of the distal region being positioned within an embodiment of an access sheath 140 having a hemostatic valve 142. As shown in the figure, the distal end 104 of the removable dual function loading tool and medical device protector 100 has a beveled configuration. Extending beyond the distal end 104 of the removable dual function loading tool and medical device protector 100 is a guide wire 32. A depth controlling rib 106 may be positioned proximate to the distal end 104 of the removable dual function loading tool and medical device protector 100. The distal end 104 allows the removable dual function loading tool and medical device protector 100 to push through the hemostatic valve 142 of the access sheath 140. As shown, the depth controlling rib 106 limits the distance that the removable dual function loading tool and medical device protector 100 can enter the access sheath. Once the removable dual function loading tool and medical device protector 100 engages the hemostatic valve 142 of the access sheath 140 and enters into the access sheath 140, a user may advance the balloon 24 of the balloon catheter 20 into the access sheath 140, and ultimately the patient, without engaging the hemostatic valve 142 of the access sheath 140. Accordingly, the removable dual function loading tool and medical device protector 100 allows for simultaneous insertion of the balloon into the access sheath 140 while also providing necessary protection to the balloon 24 prior to entry into the patient and the target site. Thus, damage to the uniformity of the coating 26 on the balloon 24 is minimized or eliminated, which may improve therapeutic efficacy of the treatment.

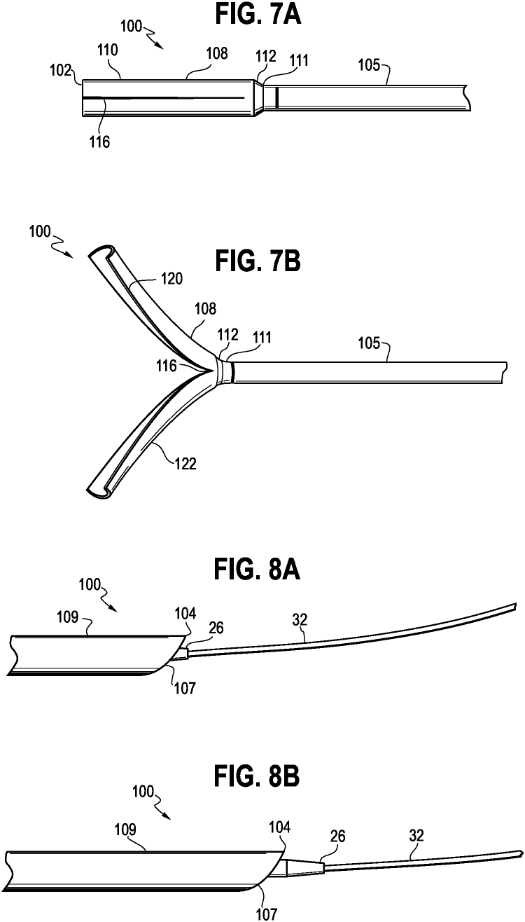

FIGS. 7A and 7B illustrate an embodiment of a proximal region 108 of the removable dual function loading tool and medical device protector 100. Referring to FIG. 7A, a proximal region 108 extends from the proximal end 102 of the body 101 of the removable dual function loading tool and medical device protector 100. A boundary 111 is present to delineate the proximal region 108 from an intermediate region 105 of the removeable dual function loading tool and medical device protector 100. The proximal region 108 includes a first section 110 and a tapered second section 112. The proximal region 108 also includes at least one slit 116 having a length that extends from the proximal end 102 of the removable dual function loading tool and medical device protector 100. As shown, the slit 116 terminates proximal to the boundary 111 and proximal to the tapered second section of the proximal region 108. The slit 116 facilitates separating, or removing, the removable dual function loading tool and medical device protector 100 from the balloon catheter once the balloon has been introduced into access sheath and the patient.

Referring now to FIG. 7B, the proximal region 108 of the removable dual function loading tool and medical device protector 100 have been separated to create a first segment 120 and a second segment 122. In use, the proximal region 108 is gripped by the user and a radial force is applied in a direction away from the center of the proximal region 108. The separation of the proximal region 108 is facilitated by slit 116 and the removable dual function loading tool and medical device protector 100 will peel along the length of the device. In some embodiments, the removable dual function loading tool and medical device protector 100 is peelable throughout its entire length such that a user may peel the removable dual function loading tool and medical device protector 100 to the hemostatic valve of the access sheath 140. In these embodiments, the user may be able to utilize the entire working length of the balloon catheter. In addition, the user may move the removable dual function loading tool and medical device protector 100 in a proximal direction out of the hemostasis valve and then peel it apart after loading the device into the access sheath and patient. In alternative embodiments, the removable dual function loading tool and medical device protector 100 may only be peelable for a certain length.

In some embodiments, the removable dual function loading tool and medical device protector 100 may utilize a strong polymeric material, such as high density polyethylene. In these embodiments, the intermediate region 105 of the removable dual function loading tool and medical device protector 100 may include some additional features to assist with peeling for steady removal of the removable dual function loading tool and balloon protector. These features may include, but not limited to, providing notches along the length of the intermediate region 105 of the removable dual function loading tool and medical device protector 100 having a specific configuration (i.e., a generally V-shape configuration), continuous linked voids having a specific configuration (i.e., circular or rectangular), or combinations thereof to allow for uniform peeling of the intermediate region 105 of the removable dual function loading tool and medical device protector 100 by reducing the strength at the peel axis. Accordingly, these embodiments of the removable dual function loading tool and medical device protector 100 may allow the user to peel the device in one motion in a similar manner as described by FIG. 7B.

FIGS. 8A and 8B illustrate the positioning of an embodiment of a distal end of the drug delivering balloon catheter 20 within an embodiment of a removable dual function loading tool and medical device protector 100. The positioning of the removable dual function loading tool and medical device protector 100 upon the balloon catheter 20 allows for the distal end 26 of the catheter shaft 22 to be in multiple positions with respect to the distal end 104 of the body of the removable dual function loading tool and medical device protector 100. As shown in FIG. 8A, the distal region 109 extends from the distal end 104 of the body 101. The distal end 104 of the body 101 of the removable dual function loading tool and medical device protector 100 has a beveled configuration. A guide wire 32 is shown extending beyond the distal end 104 of the removable dual function loading tool and medical device protector 100. The distal end 26 of the catheter shaft 22 is shown positioned within the lumen 107 and flush with the distal end 104 of the distal region 109 of the removable dual function loading tool and medical device protector 100.

As discussed above, during manufacturing of the medical device assembly, the removable dual function loading tool and medical device protector 100 is applied over the balloon to protect the balloon from damage. The distal end 26 of the catheter shaft 22 is set to be a distance away from the distal end 104 of the distal region 109 of the removable dual function loading tool and medical device protector 100. This distance may be greater than or equal to about 2 mm. This distance allows the user to maintain visibility of the distal end 26 of the catheter shaft 22. The distance also provides the user with the ability to load the balloon catheter 20 onto the wire guide during a procedure. In order to assist with maintaining this distance during manufacturing, a distance setting tool may be used to reduce the possibility of incorrect positioning.

As shown in FIG. 8B, the distal region 109 extends from the distal end 104 of the body 101. The distal end 104 of the removable dual function loading tool and medical device protector 100 has a beveled configuration. A guide wire 32 is shown extending beyond the distal end 104 of the body 101 of the removable dual function loading tool and medical device protector 100. The distal end 26 of the catheter shaft 22 is shown positioned distal to the distal region 109 and the distal end 104 of the body 101 of the removable dual function loading tool and medical device protector 100. Thus, the balloon catheter 20 is capable of movement independent of the removable dual function loading tool and medical device protector 100. This freedom of movement provides the user with flexibility with positioning of the balloon catheter 20 while manipulating the removable dual function loading tool and medical device protector 100.

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims.

One skilled in the art will realize that a virtually unlimited number of variations to the above descriptions are possible, and that the examples and the accompanying figures are merely to illustrate one or more examples of implementations.

It will be understood by those skilled in the art that various other modifications can be made, and equivalents can be substituted, without departing from claimed subject matter. Additionally, many modifications can be made to adapt a particular situation to the teachings of claimed subject matter without departing from the central concept described herein. Therefore, it is intended that claimed subject matter not be limited to the particular embodiments disclosed, but that such claimed subject matter can also include all embodiments falling within the scope of the appended claims, and equivalents thereof.

In the detailed description above, numerous specific details are set forth to provide a thorough understanding of claimed subject matter. However, it will be understood by those skilled in the art that claimed subject matter can be practiced without these specific details. In other instances, methods, devices, or systems that would be known by one of ordinary skill have not been described in detail so as not to obscure claimed subject matter.

What is claimed is:

1. A removable loading tool and protector for a medical device, comprising:
a body having a generally tubular configuration and comprising a proximal end, a distal end, an intermediate region disposed between the proximal end and the distal end, and a lumen disposed therethrough; and
a proximal region having a length and extending from the proximal end of the body, the proximal region comprising at least one slit positioned adjacent to the proximal end of the body;
wherein the proximal region has a first inner diameter and the intermediate region has a second inner diameter, the first inner diameter of the proximal region being greater than the second inner diameter of the intermediate region.

2. The removable loading tool and protector of claim 1, wherein the first inner diameter of the proximal region is uniform throughout its length.

3. The removable loading tool and protector of claim 1, wherein the first inner diameter of the proximal region varies throughout its length.

4. The removable loading tool and protector of claim 1, wherein the proximal region comprises a first section having an inner diameter and a tapered second section, the tapered second section positioned distal to the uniform first section.

5. The removable loading tool and protector of claim 4, wherein the inner diameter of the first section of the proximal region is equal to the first inner diameter of the proximal region and is uniform throughout its length.

6. The removable loading tool and protector of claim 4, wherein the tapered second section forms an angle of 45 degrees or less with respect to a longitudinal axis of the body.

7. The removable loading tool and protector of claim 6, wherein the tapered second section forms an angle of 30 degrees with respect to a longitudinal axis of the body.

8. The removable loading tool and protector of claim 1, wherein the distal end has a beveled configuration having an angle with respect to a longitudinal axis.

9. The removable loading tool and protector of claim 8, wherein the angle of the beveled configuration of the distal end ranges from 10 to 90 degrees.

10. The removable loading tool and protector of claim 1, wherein a rib is disposed about an outer surface of the body proximate to the distal end.

11. The removable loading tool and protector of claim 1, wherein the slit has a length and the length of the slit is less than the length of the proximal region.

12. A medical device assembly comprising:
a catheter having a proximal end, a distal end, and a lumen therethrough;
a drug coated expandable medical device disposed about a distal portion of the catheter, the expandable medical device having a distal end and a proximal end; and
a loading tool and protector removably disposed about the catheter and the medical device, the loading tool and protector comprising,
a body having a generally tubular configuration and comprising a proximal end, a distal end, an intermediate region disposed between the proximal end and the distal end, and a lumen disposed therethrough; and
a proximal region having a length and extending from the proximal end of the body, the proximal region comprising at least one slit positioned adjacent to the proximal end of the body;
wherein the proximal region has a first inner diameter and the intermediate region has a second inner diameter, the first inner diameter of the proximal region being greater than the second inner diameter of the intermediate region.

13. The medical device assembly of claim 12, wherein the expandable medical device is a balloon.

14. The medical device assembly of claim 12, wherein the first inner diameter of the proximal region of the loading tool and protector is uniform throughout its length.

15. The medical device assembly of claim 12, wherein the first inner diameter of the proximal region of the loading tool and protector varies throughout its length.

16. The medical device assembly of claim 12, wherein the proximal region of the loading tool and protector comprises a first section having an inner diameter and a tapered second section, the tapered second section positioned distal to the uniform first section.

17. The medical device assembly of claim 16, wherein the tapered region of the proximal region of the loading tool and protector forms an angle of 45 degrees or less with respect to a longitudinal axis of the body.

18. The medical device assembly of claim 12, wherein the distal end of the loading tool and protector has a beveled configuration having an angle.

19. The medical device assembly of claim 12, wherein a rib is disposed about an outer surface of the body of the loading tool and protector proximate to the distal end.

20. A medical device assembly comprising:
a catheter having a proximal end, a distal end, and a lumen therethrough;
a drug coated expandable balloon disposed about a distal portion of the catheter, the expandable balloon having a distal end and a proximal end; and
a loading tool and protector removably disposed about the catheter and the expandable balloon, the loading tool and protector comprising,
a body having a generally tubular configuration and comprising a proximal end, a beveled distal end, an intermediate region disposed between the proximal end and the distal end, and a lumen disposed therethrough; and
a proximal region having a length and extending from the proximal end of the body, the proximal region comprising a first section, a tapered second section, and at least one slit positioned adjacent to the proximal end of the body and within the first section, the slit having a length; and
wherein the first section of the proximal region has a first inner diameter and the intermediate region has a second inner diameter, the first inner diameter of the first section of the proximal region being greater than the second inner diameter of the intermediate region, and
wherein the length of the slit is less than the length of the proximal region.
